(19) United States
(12) Patent Application Publication
(10) Pub. No.: US 2017/0059581 A1
(43) Pub. Date: Mar. 2, 2017

(54) METHODS FOR DIAGNOSIS AND PROGNOSIS OF INFLAMMATORY BOWEL DISEASE USING CYTOKINE PROFILES

(71) Applicants: THE JOHNS HOPKINS UNIVERSITY, Baltimore, MD (US); Oklahoma Medical Research Foundation, Oklahoma City, OK (US)

(72) Inventors: Xuhang Li, Clarksville, MD (US); Philip J. Alex, Oklahoma City, OK (US); Michael B. Centola, Oklahoma City, OK (US); Nicholas Knowlton, Choctaw, OK (US)

(21) Appl. No.: 15/258,050

(22) Filed: Sep. 7, 2016

Related U.S. Application Data
(63) Continuation of application No. 15/189,490, filed on Jun. 22, 2016.
Application No. 15/189,490 is a continuation of application No. 13/823,159, filed on Jun. 24, 2013, now abandoned.
(60) Provisional application No. 61/382,549, filed on Sep. 14, 2010.
(60) Provisional application No. 61/437,269, filed on Jan. 28, 2011.

(51) Int. Cl. G01N 33/68

(52) U.S. Cl. CPC G01N 33/6863; G01N 33/6866; G01N 33/6869; G01N 2333/535; G01N 2333/522; G01N 2333/521; G01N 2800/60; G01N 2800/065; G01N 2333/57; G01N 2333/55; G01N 2333/5403; G01N 2333/5406; G01N 2333/545; G01N 2333/5409; G01N 2333/5412; G01N 2333/5421; G01N 2333/5428; G01N 2333/5434

(57) ABSTRACT

The present invention relates to the field of inflammatory bowel disease. More specifically, the present invention relates to the use of cytokines to detect, diagnose, and assess inflammatory bowel disease. In one embodiment, a method for diagnosing Crohn's Disease (CD) in a patient comprises the steps of (a) collecting a sample from the patient; (b) measuring the levels of at least one cytokine in the sample collected from the patient; and (c) comparing the levels of the at least one cytokine with predefined cytokine levels, wherein a correlation between the cytokine levels in the patient sample and predefined cytokine levels indicates that the patient has CD. In a specific embodiment, the at least one cytokine comprises Interferon (IFN)-gamma, Interleukin (IL)-1beta, IL-6, IL-8, IL-12, IL-17 and CXCL10.

### CROSS-REFERENCE TO RELATED APPLICATIONS

This application claims the benefit of U.S. Provisional Application No. 61/437,269, filed Jan. 28, 2014, and U.S. Provisional Application No. 61/382,549, filed Sep. 14, 2010, both of which are incorporated herein by reference in their entireties.

### FIELD OF THE INVENTION

The present invention relates to the field of inflammatory bowel disease. More specifically, the present invention relates to the use of cytokines to detect, diagnose, and assess inflammatory bowel disease.

### BACKGROUND OF THE INVENTION

With a prevalence of about 0.2% Western population, inflammatory bowel disease (IBD), primarily consisting of two forms: Crohn's Disease (CD) and Ulcerative Colitis (UC), and a less frequent form, Indeterminate Colitis (IC), is a chronic, progressive, and systemic, autoimmune inflammatory disorder of the gastrointestinal tract. See Strober et al., 117 J. CLIN. INVEST. 514-21 (2007); Xavier et al., 448 NATURE 427-34 (2007); Sartor, R. B., 3 NAT. CLIN. PRACT. GASTROENTEROL. HEPATOL., 390-407 (2006); and Geboes et al., 9 INFLAMM. BOWEL DIS. 324-31 (2003). According to Crohn's & Colitis Foundation of America (CCFA), in the United States alone, there are over 1.4 million diagnosed IBD patients, with approximately 30,000 new cases diagnosed each year. With increasing prevalence all over the world, IBD has enormous suffering, morbidity, and health-care costs, and increases the risk for colorectal cancer. See Xavier et al., 448 NATURE 427-34 (2007); and Jess et al., 12 INFLAMM. BOWEL DIS. 669-76 (2006).

Inflammatory responses in IBD are pathologic features driven by cytokine and chemokine mechanisms. See Nishimura et al., 1173 ANN. N. Y. ACAD. SCI. 350-56 (2009); Andoh et al., 14 WORLD J. GASTROENTEROL. 5154-61 (2008); Kolls et al., 8 NAT. REV. IMMUNOL. 829-35 (2008); Sanchez-Munoz et al., 14 WORLD J. GASTROENTEROL. 4280-88 (2008); and Fantini et al., 13 INFLAMM. BOWEL DIS. 1419-23 (2007). Cytokines are defined as any of several regulatory proteins, such as the interleukins and lymphokines, that are released by cells of the immune system and act as intercellular mediators in the generation of an immune response. See Bettelli et al., 453 NATURE 1051-57 (2008); O'Shea et al., 28 IMMUNITY 477-87 (2008); and Furuzawa-Carballeda et al., 6 AUTOIMMUNE REV. 169-75 (2007). Cytokines are secreted by immune or other cells, whose action are on cells of the immune system, such as, but not limited to, T-cells, B-cells, NK cells and macrophages. Chemokines are defined as chemotactic cytokines produced by a variety of cell types in acute and chronic inflammation that mobilize and activate while blood cells. Charo et al., 354 N. ENGL. J. MED. 610-21 (2006); and Zlotnik et al., 7 GENOME BIOL. 243 (2006). Cytokines and chemokines are important cell signaling proteins, mediating a wide range of physiological and pathological responses, including immunity, inflammation, and hematopoiesis. See Kurtz et al., 2009 MEDIATORS INFLAMM. 1-20 (2009); Pizarro et al., 58 ANN. REV. MED. 433-44 (2007); and Pizarro et al., 55 GUT 1226-27 (2006).

Several therapeutic agents, primarily directed at cytokines, are currently available and have shown great promise in the treatment of IBD. See Dryden, G. W., 9 EXPERT OPIN. BIOL. THER. 967-74 (2009); and Rutgeerts et al., 136 GASTEROLOGY 1182-97 (2009). While previous studies have been done to evaluate systemic cytokine profiles in IBD, they have been limited to a relatively small number of cytokines and the analysis of absolute level of each cytokine without taking into account the interplay of multiple cytokines. Li et al., 14 WORLD J. GASTOENTEROL. 5115-24 (2008). There are no tests or indications available of whether patients have the specific cytokine antagonized by the therapeutic agent, or whether patients will positively respond to the medications. Furthermore, despite the need to identify cytokine associations with IBD, there has been no definitive link identified between cytokine levels and diagnosis, prognosis, and treatment response of such pathologic states.

### SUMMARY OF THE INVENTION

The present invention is based, in part, on the discovery that unique profiles of cytokines/chemokines can be used to differentiate inflammatory bowel disease phenotypes and severity. As described herein, the inventors determined the relevant cytokine profiles from sera of patients with IBD. Distinct disease-specific cytokine profiles were identified respectively in CD, UC, IC, and relative to healthy controls. Profiles were found to have significant correlations to disease activity and duration of disease.

Cytokine profiles that were identified at the systemic level using immunoassays were also validated locally in colonic mucosa tissues using immunoblotting methods, immunofluorescence assays, and chemiluminescence assays. Furthermore, results obtained from patients with IBD were also validated using murine models of IBD utilizing immunoassays, immunoblotting methods, immunofluorescence assays, immunostaining methods, and chemiluminescence assays. Advanced multivariate analyses including cluster analysis; factor analysis; canonical analysis; linear and non-linear mapping techniques; regression analyses; discriminant function analysis; and pattern analysis including principal component analysis, multidimensional scaling, probabilistic methods, and dynamic neural networks were used to provide detailed characterization of cytokine-based IBD subtypes.

These methods and analytical tools were utilized to identify novel diagnostic discriminatory cytokine biomarkers that can be sufficiently used to distinguish one IBD disease subtype from each other and controls. Furthermore, these tools were utilized to develop innovative diagnostic, prognostic, disease activity-based, predictive, and therapeutic response panel of markers in patients with IBD disease subtypes.

This is the first time that profiling, as applied particularly to cytokines, has been used to diagnose, and correlate with disease activity in IBD. Accordingly, in one aspect, the present invention provides methods for diagnosing IBD. In one embodiment, a method for diagnosing Crohn's Disease (CD) in a patient comprises the steps of (a) collecting a sample from the patient; (b) measuring the levels of at least one cytokine in the sample collected from the patient; and (c) comparing the levels of the at least one cytokine with predefined cytokine levels, wherein a correlation between the cytokine levels in the patient sample and predefined cytokine levels indicates that the patient has CD. In a specific embodiment, the at least one cytokine is selected from the group consisting of Interferon (IFN)-γ, Interleukin (IL)-1β, IL-6, IL-8, IL-12, IL-17 and CXCL10. In a more specific embodiment, the at least one cytokine comprises Interferon (IFN)-γ, Interleukin (IL)-1β, IL-6, IL-8, IL-12, IL-17 and CXCL10. In a particular embodiment, a method for diagnosing CD in a patient comprises the steps of (a) collecting a sample from the patient; (b) measuring the levels of IKN-γ, IL-1β, IL-6, IL-8, IL-12, IL-17 and CXCL10 in the sample collected from the patient; and (c) comparing the levels with predefined cytokine levels, wherein a correlation between the cytokine levels in the patient sample and predefined cytokine levels indicates that the patient has CD.

In another embodiment, a method for diagnosing Ulcerative Colitis (UC) in a patient comprises the steps of (a) collecting a sample from the patient; (b) measuring the levels of at least one cytokine in the sample collected from the patient; and (c) comparing the levels of the at least one cytokine with predefined cytokine levels, wherein a correlation between the cytokine levels in the patient sample and predefined cytokine levels indicates that the patient has UC. In a specific embodiment, the at least one cytokine is selected from the group consisting of IL-5, IL-10, Granulocyte-Colony Stimulating Factor (G-CSF), IL-1F3, and Eotaxin (CCL-11). In a more specific embodiment, the at least one cytokine comprises IL-5, IL-10, G-CSF, IL-1F3, and Eotaxin. In a particular embodiment, a method for diagnosing UC in a patient comprises the steps of (a) collecting a sample from the patient; (b) measuring the levels of IL-5, IL-10, G-CSF, IL-1F3, and Eotaxin in the sample collected from the patient; and (c) comparing the levels with predefined cytokine levels, wherein a correlation between the cytokine levels in the patient sample and predefined cytokine levels indicates that the patient has UC.

In a further embodiment, a method for diagnosing Indeterminate Colitis (IC) in a patient comprises the steps of (a) collecting a sample from the patient; (b) measuring the levels of at least one cytokine in the sample collected from the patient; and (c) comparing the levels of the at least one cytokine with predefined cytokine levels, wherein a correlation between the cytokine levels in the patient sample and predefined cytokine levels indicates that the patient has IC. In a specific embodiment, the at least one cytokine is selected from the group consisting of IL-2, IL-4, IL-5, IL-17, IFN-γ, and G-CSF. In a more specific embodiment, the at least one cytokine comprises IL-2, IL-4, IL-5, IL-17, IFN-γ, and G-CSF. In a particular embodiment, a method for diagnosing IC in a patient comprises the steps of (a) collecting a sample from the patient; (b) measuring the levels of IL-2, IL-4, IL-5, IL-17, IFN-γ, and G-CSF in the sample collected from the patient; and (c) comparing the levels with predefined cytokine levels, wherein a correlation between the cytokine levels in the patient sample and predefined cytokine levels indicates that the patient has IC.

In another aspect, the present invention provides methods for determining the IBD status of a patient. In one embodiment, the methods comprises the steps of (a) collecting a sample from the patient; (b) measuring the levels of at least one cytokine in a sample collected from the patient; and (c) comparing the levels of the at least one cytokine with predefined cytokine levels that correspond to a patient not having IBD, predefined cytokine levels that correspond to a patient having CD, predefined cytokine levels that correspond to a patient having UC, and predefined cytokine levels that correspond to a patient having IC, wherein a correlation between the levels of the at least one cytokine in the sample from the patient and one of the predefined cytokine levels is indicative of the IBD status of the patient. In a specific embodiment, the at least one cytokine is selected from the group consisting of IL-1β, IL-1F3, IL-2, IL-4, IL-5, IL-6, IL-8, IL-10, IL-12, IL-17, IFN-γ, G-CSF, Exotaxin, and CXCL10. In a more specific embodiment, the at least one cytokine comprises IL-1β, IL-1F3, IL-2, IL-4, IL-5, IL-6, IL-8, IL-10, IL-12, IL-17, IFN-γ, G-CSF, Eotaxin, and CXCL10.

In certain embodiments, the patient sample comprises peripheral blood, serum plasma, cerebrospinal fluid, tissue sample, skin or other body fluid, in a specific embodiment, the patient sample comprises serum plasma. In particular embodiments, the measuring step is assessed using an immunoassay, immunoblotting method, immunoprecipitation assay, immunostaining method, immunofluorescent assay, or a chemiluminescence assay. In specific embodiments, the immunoassay is an enzyme-linked immunosorbent assay, magnetic immunoassay, or a radioimmunoassay.

### BRIEF DESCRIPTION OF THE FIGURES

FIG. 1 depicts the unique cytokine profiles from IBD patients (UC and CD) and unaffected healthy controls.

FIG. 2 illustrates the potential of cytokine profiles to discriminate between (IC), UC, and CD.

FIG. 3 provides a visual representation of the observed similarities and dissimilarities between the disease profiles of UC, CD and IC, and their associated cytokine patterns.

FIG. 4 depicts the discriminative potential of the cytokine profiles from UC, CD and relative to unaffected healthy controls.

FIG. 5 portrays the value of the cytokine profiles represented on a clinical scoring scale, the inflammatory activity index.

### DETAILED DESCRIPTION OF THE INVENTION

It is understood that the present invention is not limited to the particular methods and components, etc., described herein, as these may vary, it is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms mid phrases employed in the specification, examples, and appended claims are provided. The definitions are not meant to be limiting in nature and serve to provide a clearer understanding of certain aspects of the present invention.

### I. Definitions

As used herein, the term “comparing” refers to making an assessment of how the proportion, level or cellular localization of one or more cytokines in a sample from a patient relates to the proportion, level or cellular localization of the corresponding one or more cytokines in a standard or control sample. For example, “comparing” may refer to assessing whether the proportion, level, or cellular localization of one or more cytokines in a sample from a patient is the same as, more or less than, or different from the proportion, level, or cellular localization of the corresponding one or more cytokines in standard or control sample. More specifically, the term may refer to assessing whether the proportion, level, or cellular localization of one or more cytokines in a sample from a patient is the same as, more or less than, different from or otherwise corresponds (or not) to the proportion, level, or cellular localization of predefined cytokine levels that correspond to, for example, a patient not having IBD, having/not having-CD, having/not having UC, having/not having IC or having/not having another disease or condition.

As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient has IBD (e.g., one of UC, CD or IC). In specific embodiments, the parameter may comprise the presence, absence and/or particular amounts of one or more cytokines of the present invention. In other embodiments a parameter may comprise a weight in a multivariate algorithm (e.g., BOOSTED models, C&RT, Random Forests, Penalized regression models). The term “pattern” may mean a multivariate algorithm. A particular set or pattern of one or more cytokines (including the presence, absence, and/or particular amounts) may indicate that a patient has IBD (or correlates to a patient having IBD), in particular, UC. In other embodiments, a particular set or pattern of one or more cytokines (including the presence, absence, and/or particular amounts) may be correlated to a patient having CD (or may indicate that a patient has CD). In other embodiments, a particular set or pattern of one or more cytokines (including the presence, absence, and/or particular amounts) may be correlated to a patient having IC (or may indicate that a patient has IC). In yet other embodiments, a particular set or pattern of one or more cytokines (including the presence, absence, and/or particular amounts) may be correlated to a patient being unaffected. In certain embodiments, “indicating,” or “correlating,” as used according to the present invention, may be by any linear or non-linear method of quantifying the relationship between levels of expression or localization of markers to a standard, control or comparative value for the assessment of the diagnosis, prediction of an IBD or IBD progression, assessment of efficacy of clinical treatment, identification of a patient that may respond to a particular treatment regime or pharmaceutical agent, monitoring of the progress of treatment, and in the context of a screening assay, for the identification of an anti-IBD (CD, UC, or IC) therapeutic.

The terms “patient,” “individual” or “subject” are used interchangeably herein, and refer to a mammal, particularly, a human. The patient may have mild, intermediate or severe disease. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters, and primates.

The terms “measuring” and “determining” are used interchangeably throughout, and refer to methods which include obtaining a patient sample, detecting the presence or absence of a cytokine(s) in a sample, quantifying the amount of cytokine(s) in the sample, and/or qualifying the type of cytokine(s). In one embodiment, the terms refer to obtaining a patient sample and detecting the presence, absence, and or particular amounts of one or more cytokines in the sample. In another embodiment, the terms “measuring” and “determining” mean detecting the presence, absence, and/or particular amounts of one or more cytokines in a patient sample. Measuring can be accomplished by methods known in the art and those further described herein including, but not limited to, immunoassay and mass spectrometry. The term “measuring” is also used interchangeably throughout with the term “detecting.”

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms of IBD, and/or extra-intestinal involvement. Moreover, a sample obtained from a patient can be divided and only a portion may be used to for diagnosis. Further, the sample, or a portion thereof, can be stored under conditions to maintain sample for later analysis. The definition specifically encompasses blood and other liquid samples of biological origin (including, but not limited to peripheral blood, serum, plasma, urine, saliva, stool and synovial fluid), solid tissue samples such as a biopsy specimen or tissue cultures or cells derived therefrom and the progeny thereof. In a specific embodiment, a sample comprises a blood sample. In another embodiment, a serum sample is used. The definition also includes samples that have been manipulated in any way after their procurement, such as by centrifugation, nitration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

Various methodologies of the instant invention include a step that involves comparing a value, level, feature, characteristic, property, etc. to a “suitable control,” referred to interchangeably herein as an “appropriate control” or a “control sample.” A “suitable control,” “appropriate control” or a “control sample” is any control or standard familiar to one of ordinary skill in the art useful for comparison purposes. In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the cytokines of the present invention may be assayed for their presence, absence and/or particular amounts in a sample from an unaffected individual (UI) or a normal control individual (NC) (both terms are used interchangeably herein). In another embodiment, a “suitable control” or “appropriate control” is a value, level, feature, characteristic, property, etc. determined prior to performing a therapy (e.g., an IBD treatment) on a patient. In yet another embodiment, a transcription rate, mRNA level, translation rate, protein level, biological activity, cellular characteristic or property, genotype, phenotype, etc. can be determined prior to, during, or after administering a therapy into a cell, organ, or patient. In a further embodiment, a “suitable control” or “appropriate control” is a predefined value, level, feature, characteristic, property, etc.

### II. Detection of Cytokine Biomarkers

A. Detection by Mass Spectrometry

In one aspect, the cytokines of the present invention may be detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer, hybrids or combinations of the foregoing, and the like. In a specific embodiment, the mass spectrometric method comprises matrix assisted laser desorption/ionization time-of-flight (MALDI-TOF MS or MALDI-TOF). In another embodiment, method comprises MALDI-TOF tandem mass spectrometry (MALDI-TOF MS/MS). In yet another embodiment, mass spectrometry can be combined with another appropriate method(s) as may be contemplated by one of ordinary skill in the art. For example, MALD-TOF can be utilized with trypsin digestion and tandem mass spectrometry as described herein.

In an alternative embodiment, the mass spectrometry technique comprises surface enhanced laser desorption and ionization or “SELDI,” as described, for example, in U.S. Pat. No. 6,225,047 and No. 5,719,060. Briefly, SELDI refers to a method of desorption/ionization gas phase ion spectrometry (e.g. mass spectrometry) in which an analyte (here, one or more of the biomarkers) is captured on the surface of a SELDI mass spectrometry probe. There are several versions of SELDI that may be utilized including, but not limited to, Affinity Capture Mass Spectrometry (also called Surface-Enhanced Affinity Capture (SEAC)), and Surface-Enhanced Neat Desorption (SEND) which involves the use of probes comprising energy absorbing molecules that are chemically bound to the probe surface (SEND probe). Another SELDI method is called Surface-Enhanced Photolabile Attachment and Release (SEPAR), which involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker panel, pursuant to the present invention.

In another mass spectrometry method, the biomarkers can be first captured on a chromatographic resin having chromatographic properties that bind the biomarkers. For example, one could capture the cytokines on a cation exchange resin, such as CM Ceramic HyperD F resin, wash the resin, elute the cytokines and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the cytokine biomarkers on an immuno-chromatographic resin that comprises antibodies that bind the cytokines, wash the resin to remove unbound material, elute the cytokines from the resin and detect the eluted cytokines by MALDI or by SELDI.

B. Detection by Immunoassay

In other embodiments, the cytokines of the present invention can be detected and/or measured by immunoassay. Immunoassay requires biospecific capture reagents, such as antibodies, to capture the cytokines. Many antibodies are available commercially. Antibodies also can be produced by methods well known in the art, e.g., by immunizing animals with the cytokines. Cytokines can be isolated from samples based on their binding characteristics. Alternatively, if the amino acid sequence of a polypeptide cytokine is known, the polypeptide can be synthesized and used to generate antibodies by methods well-known in the art.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the cytokine is attached to the surface of an MS probe, such as a pre-activated ProteinChip array. The cytokine is then specifically captured on the biochip through this reagent, and the captured cytokine is detected by mass spectrometry. The Quantikine immunoassay developed by R&D Systems, Inc. (Minneapolis, Minn.) may also be used in the methods of the present invention.

C. Detection by Electrochemicaluminescent Assay

In several embodiments, the cytokine biomarkers of the present invention may be detected by means of an electrochemicaluminescent assay developed by Meso Scale Discovery (Gaithersburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. No. 7,497,097; No. 7,491,540; No. 7,288,410; No. 7,036,946; No. 7,052,861; No. 6,977,722; No. 6,919,173; No. 6,673,533; No. 6,413,783; No. 6,362,011; No. 6,319,670; No. 6,207,369; No. 6,140,045; No. 6,090,545; and No. 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

D. Other Methods for Detecting Cytokine Biomarkers

The cytokines of the present invention can be detected by other suitable methods. Detecting paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemilumineseence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Invitrogen Corp. (Carlsbad, Calif.), Affymetrix, Inc. (Fremong, Calif.), Zyomyx (Hayward, Calif.), R&D Systems, Inc. (Minneapolis, Minn.), Biacore (Uppsala, Sweden) and Procognia (Berkshire, UK). Examples of such protein biochips are described in the following patents or published patent applications: U.S. Pat. No. 6,537,749; U.S. Pat. No. 6,329,209; U.S. Pat. No. 6,225,047; U.S. Pat. No. 5,242,828; PCT International Publication Mo. WO 00/56934; and PCT International Publication No. WO 03/048768.

### III. Determination of Patient Inflammatory Bowel Disease Status

The present invention relates to the use of cytokines to detect IBD. More specifically, the cytokines of the present invention can be used in diagnostic tests to determine, qualify, and/or assess IBD status, for example, to diagnose IBD, in an individual, subject or patient. In one aspect, the present invention provides cytokine panels for discriminating among individuals with Crohn's Disease (CD), individuals with Ulcerative Colitis (UC), individuals with Indeterminate Colitis (IC) and unaffected individuals (UI) (also referred to herein as normal control individuals (NC)). In certain embodiments, the present invention can be used to distinguish among CD, UC and IC in an individual.

More specifically, the cytokines to be detected in diagnosing UC include, but are not limited to, Interleukin (IL)-5, IL-10, Granulocyte-Colony Stimulating Factor (G-CSF), IL-1F3, and Eotaxin (CCL-11). The cytokines to be detected in diagnosing CD include, but are not limited to, Interferon (IFN)-γ, IL-1β, IL-6, IL-8, IL-12, IL-17 and CXCL10. The cytokines to be detected in diagnosing IC include, but are not limited to, IL-2, IL-4, IL-5, IL-17, IFN-γ, and G-CSF. In particular embodiments, a patient sample is tested for the presence, absence and/or particular amounts of IL-1β, IL-1F3, IL-2, IL-4, IL-5, IL-6, IL-8, IL-10, IL-12, IL-17, IFN-γ, G-CSF, Eotaxin, and CXCL10.

Other cytokines known in the relevant art may be used in combination with the cytokines described herein including, but not limited to, C5, IL-32α, CD40 ligand, CXCL11/I-TAC, GM-CSF, IL-8, CCLs/MCP-1, CXCL1/I-309, IL-12p70, CCL3/MIL-1α, ICAM-1, IL-13, CCL4/MIP-1β, CCL5/RANTES, IL-1α, CXCL12/SDF-1, IL-17E, Serpin E1/PAI-1, IL-1ra, IL-23, TNF-alpha, IL-27, and TREM-1.

A. Cytokine Panels

The cytokines of the present invention can be used m diagnostic tests to assess, determine, and/or qualify (used interchangeably herein) IBD status in a patient. The phrase “IBD status” includes any distinguishable manifestation of the disease, including non-disease. For example, IBD status includes, without limitation, the presence or absence of IBD (e.g., distinguishing between UI and IBD (UC, CD, or IC) in a patient), the risk of developing IBD (e.g., distinguishing between UI and IBD in a patient or distinguishing among UC, CD and IC in a patient), the stage of IBD, the progress of IBD (e.g., progress of IBD over time) and the effectiveness or response to treatment of IBD (e.g., clinical, follow up and surveillance of UC, CD or IC after treatment). Based on this status, further procedures may be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments, the cytokine panels of the present invention may show a statistical difference in different IBD statuses of at least p<0.05, p<10⁻², p<10⁻³, p<10⁻⁴ or p<10⁻⁵. Diagnostic tests that use these cytokines may show an ROC of at least 0.6, at least about 0.7, at least about 0.8, or at least 0.9 about.

The cytokines are differentially present in UI (or NC), UC, CD, and IC, and, therefore, are useful in aiding in the determination of IBD status. In certain embodiments, the cytokines are measured in a patient sample using the methods described herein and compared, for example, to predefined cytokine levels and correlated to IBD status. In particular embodiments, the measurement(s) may then be compared with a relevant diagnostic amount(s), cut-off(s), or multivariate model scores that distinguish a positive IBD status from a negative IBD status. The diagnostic amount(s) represents a measured amount of a cytokine(s) above which or below which a patient is classified as having a particular IBD status. For example, if the cytokine(s) is/are up-regulated compared to normal during IBD, then a measured amount(s) above the diagnostic cut-off(s) provides a diagnosis of IBD. Alternatively, if the cytokine(s) is/are down-regulated during IBD, then a measured amount(s) below the diagnostic cutoff(s) provides a diagnosis of IBD. As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the amount of the cytokine(s) in a statistically significant number of samples from patients with the different IBD statuses, and drawing the cut-off to suit the desired levels of specificity and sensitivity.

Indeed, as the skilled artisan will appreciate there are many ways to use the measurements of two or more markers in order to improve the diagnostic question under investigation. In a quite simple, but nonetheless often effective approach, a positive result is assumed if a sample is positive for sit least one of the markers investigated.

Furthermore, in certain embodiments, the values measured for markers of a cytokine panel are mathematically combined and the combined value is correlated to the underlying diagnostic question. Cytokine values may be combined by any appropriate state of the art mathematical method. Well-known mathematical methods for correlating a marker combination to a disease employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate a cytokine combination of the present invention. In one embodiment, the method used in correlating cytokine combination of the present invention, e.g. to diagnose IBD, is selected from DA (e.g., Linear-, Quadratic-, Regularized Discriminant Analysis), DFA, Kernel Methods (e.g., SVM), MDS, Nonparametric Methods (e.g., k-Nearest-Neighbor (Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastic, Trevor, Tibshirani, Robert, Friedman, Jerome, The Elements of Statistical Learning, Springer Series in Statistics (2001); Breiman, I., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

B. Determining Risk of Developing IBD

In a specific embodiment, the present invention provides methods for determining the risk of developing IBD in a patient. Cytokine amounts or patterns are characteristic of various risk states, e.g., high, medium or low. The risk of developing IBD is determined by measuring the relevant cytokines and then either submitting them to a classification algorithm or comparing them with a reference amount, i.e., a predefined level or pattern of cytokines that is associated with the particular risk level. Further embodiments include determining if a patient has or will develop an inflammatory or autoimmune disease.

C. Determining IBD Severity

In another embodiment, the present invention provides methods for determining the severity of IBD in a patient. Each stage of IBD—mild, intermediate or severe—has a characteristic amount of a cytokine or relative amounts of a set of cytokines (a pattern). The severity of IBD is determined by measuring the relevant cytokines and then either submitting them to a classification algorithm or comparing them with a reference amount, i.e., a predefined level or pattern of cytokines that is associated with the particular stage.

D. Determining IBD Prognosis

In one embodiment, the present invention provides methods (or determining the course of IBD in a patient. IBD course refers to changes in IBD status over time, including IBD progression (worsening) and IBD regression (improvement). Over time, the amounts or relative amounts (e.g., the pattern) oft be cytokines change. For example, cytokine “X” may be increased with CD, while cytokine “Y” may be decreased in CD. Therefore, the trend of these cytokines, either increased or decreased overtime toward IBD or non-IBD indicates the course of the disease. Accordingly, this method involves measuring one or more cytokines in a patient at least two different time points, e.g., a first time and a second time, and comparing the change in amounts, if any. The course of IBD is determined based on these comparisons.

E. Patient Management

In certain embodiments of the methods of qualifying IBD status, the methods further comprise managing patient treatment based on the status. Such management includes the actions of the physician or clinician subsequent to determining IBD status. For example, if a physician makes a diagnosis of CD, then a certain regime of monitoring would follow. An assessment of the course of CD using the methods of the present invention may then require a certain IBD therapy regimen. Alternatively, a diagnosis of non-IBD might be followed with further testing to determine a specific disease that the patient might be suffering from. Also, further tests may be called for if the diagnostic test gives an inconclusive result on IBD status.

F. Determining Therapeutic Efficacy of Pharmaceutical Drug

In another embodiment, the present invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of one or more of the cytokines of the present invention may change toward a non-IBD profile. Therefore, one can follow the course of the amounts of one or more cytokines in the patient during the course of treatment. Accordingly, this method involves measuring one or more cytokines in a patient receiving drug therapy, and correlating the amounts of the cytokines with the IBD status of the patient (e.g., by comparison to predefined cytokine levels that correspond to different IBD statuses). One embodiment of this method involves determining the levels of one of more cytokines at least two different time points during a course of drug therapy, e.g., a first time and a second time, and comparing the change in amounts of the cytokines, if any. For example, the one or more cytokines can be measured before and after drug administration or at two different time points during drug administration. The effect of therapy is determined based on these comparisons. If a treatment is effective, then one or more cytokines will trend toward normal, while if treatment is ineffective, the one or more cytokines will trend toward IBD indications.

G. Generation of Classification Algorithms for Qualifying IBD Status

In some embodiments, data that are generated using samples such as “known samples” can then be used to “train” a classification model. A “known sample” is a sample that has been pre-classified. The data that are used to form the classification model can be referred to as a “training data set.” The training data set that is used to form the classification model may comprise raw data or pre-processed data. Once trained, the classification model can recognize patterns in data generated using unknown samples. The classification model can then be used to classify the unknown samples into classes. This can be useful, for example, in predicting whether or not a particular biological sample is associated with a certain biological condition (e.g., diseased versus non-diseased).

Classification models can be formed using any suitable statistical classification or learning method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART), artificial neural networks such as back propagation networks, discriminant analyses (e.g., Bayesian classifier or Fischer analysis), logistic classifiers, and support vector classifiers (support vector machines).

Another supervised classification method is a recursive partitioning process. Recursive partitioning processes use recursive partitioning trees to classify data derived from unknown samples. Further details about recursive partitioning processes are provided in U.S. Patent Application No. 2002 0138208 A1 to Paulse el al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application Publication No. 2002/0193950 (Gavin et al. “Method or analyzing mass spectra”), U.S. Patent Application Publication No. 2003/0004402 (Hitt et al., “Process for discriminating between biological states based on hidden patterns from biological data”), and U.S. Patent Application Publication No. 2003/0055615 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows® or Linux™ based operating system. In embodiments utilizing a mass spectrometer, the digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including R, C, C++, visual basic, etc.

The learning algorithms described above are useful both for developing classification algorithms for the cytokine biomarkers already discovered, and for finding new cytokine biomarkers. The classification algorithms, in turn, form the base for diagnostic tests by providing diagnostic values (e.g., cut-off points) for cytokines used singly or in combination.

H. Kits for the Detection of IBD Cytokine Biomarkers

In another aspect, the present invention provides kits for qualifying IBD status, which kits are used to detect the cytokines described herein. In a specific embodiment, the kit is provided as an ELISA kit comprising antibodies to the cytokines of the present invention including, but not limited to, IL-1β, IL-1F3, IL-2, IL-4, IL-5, IL-6, IL-8, IL-10, IL-12, IL-17, IFN-γ, G-CSF, Eotaxin, and CXCL10.

The ELISA kit may comprise a solid support, such as a chip, microtiter plate (e.g., a 96-well plate), bead, or resin having cytokine capture reagents attached thereon. The kit may further comprise a means for detecting the cytokines, such as antibodies, and a secondary antibody-signal complex such as horseradish peroxidase (HRP)-conjugated goat anti-rabbit IgG antibody and tetramethyl benzidine (TMB) as a substrate for HRP.

The kit for qualifying IBD status may be provided as an immuno-chromatography strip comprising a membrane on which the antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of blood serum.

A patient can be diagnosed by adding blood or blood serum from the patient to the kit and detecting the relevant cytokines conjugated with antibodies, specifically, by a method which comprises the steps of: (i) collecting blood or blood serum from the patient; (ii) separating blood serum from the patient's blood; (iii) adding the blood serum from patient to a diagnostic kit; and, (iv) detecting the cytokines conjugated with antibodies. In this method, the antibodies are brought into contact with the patient's blood. If the cytokines are present in the sample, the antibodies will bind to the sample, or a portion thereof. In other kit and diagnostic embodiments, blood or blood serum need not be collected from the patient (i.e., it is already collected). Moreover, in other embodiments, the sample may comprise a tissue sample or a clinical sample.

The kit can also comprise a washing solution or instructions for making a washing solution, in which the combination of the capture reagents and the washing solution allows capture of the cytokines on the solid support for subsequent detection by, e.g., antibodies or mass spectrometry. In a further embodiment, a kit can comprise instructions for suitable operational parameters in the form of a label or separate insert. For example, the instructions may inform a consumer about how to collect the sample, how to wash the probe or the particular cytokines to be detected. In yet another embodiment, the kit can comprise one or more containers with cytokine samples, to be used as standard(s) for calibration.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the cytokines, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Multiplex Serum Cytokine Profiling

Multiplex serum cytokine profiling from serum of IBD and controls was performed. The cohort also included unaffected age and sex matched unaffected controls. The following 24 cytokines were assessed (Invitrogen Corp., Carlsbad, Calif.): IL-1ra, IL-1α, IL-1β, IL-2, IL-4, IL-5, IL-6, IL-10, IL-12, IL-13, IL-17, IFN-γ, TNF-α, G-CSF, GM-CSF, IL-8, MIP-1α, MIP-1β, MCP-1, EGF, VEGF, FGF-basic, IP-10, and Eotaxin. A total of 151 IBD patients (69 UC and 82 CD) and 80 controls were assessed.

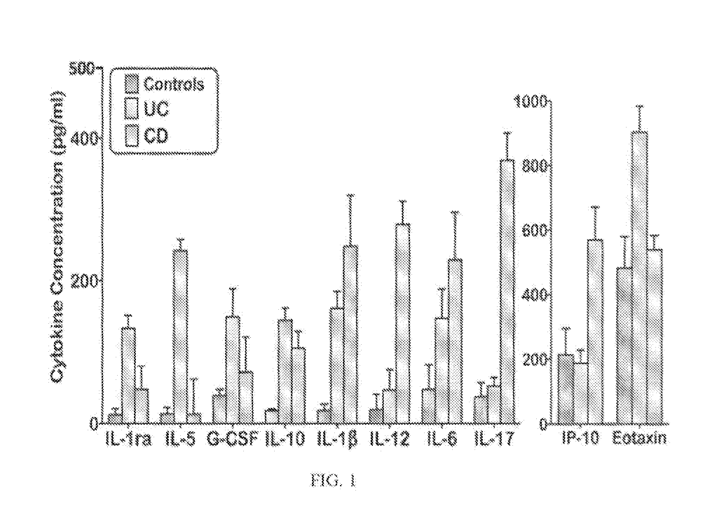

As shown in FIG. 1, UC patients demonstrated significantly lower levels of IL-1β, IL-12, IL6, IL-17, and IP-10 and significantly elevated levels of IL-1ra, IL-5, G-CSF, IL-10, and Eotaxin when compared to CD, suggesting a Th2-chemotactic biased profile in UC, and a Th1/Th17 predominant profile in CD. In addition, IL-12 and IL-17 were only found to be significantly elevated in CD, suggestive of their significance in the immunomodulatory pathogenesis and their importance as reliable serological cytokines for CD. While there are significant differences cytokine/chemokine biomarkers in human IBD when compared to murine chronic IBD, it is important to note that the immunomodulatory Th and chemokine profiles observed in our studies were consistent between both models.

### Example 2

### Cytokine Level Assessment of Patients with Indeterminate Colitis (IC)

To evaluate the potential of the cytokine profiles to discriminate indeterminate colitis from that of IBD, the cytokine levels from serum of an additional 57 patients with indeterminate colitis were further assessed. Cytokine levels from serum of IC patients were both distinctive and overlapping with that of patients with diagnosed UC and CD. A multivariate analysis called Discriminant Functional Analysis (DFA) was used for selection of the set of analytes that maximally discriminate among IC, UC, and CD built in a step-wise manner. This was then included in a discriminative function, denoted a root, which is an equation consisting of a linear combination of changes in analytes used for the prediction of group membership.

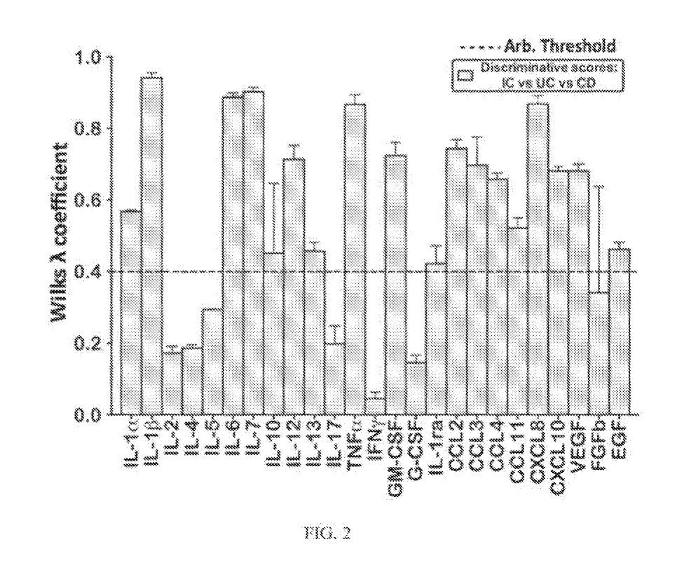

An F test was used to determine the statistical significance of the discriminatory power of the selected analytes. which was also characterized by a Wilk's lambda coefficient. This coefficient ranges from 1.0 (no discriminatory power) to 0.0 (perfect discriminatory power), and as shown in FIG. 2 was able to identify six cytokines IL-2, IL-4, IL-5, IL-17, IFNγ, and G-CSF with the power to discriminate IC, UC, and CD.

Multidimensional Scaling (MDS), which is an iterative process to detect meaningful underlying dimensions to explain observed similarities or dissimilarities between the groups studied, was also used. This analysis uses correlational matrices to construct configurations of the data in a lower dimensional matrix, such that the relative distances between the groups are similar to those in the higher dimensional matrix. The degree of correspondence between the distances and the matrix input by the user is measured (inversely) by a stress function defined by Phi=Σ[dij−f(δij)]2, where dij stands for the euclidean distance, and δij stands for the observed distance. The proximities and distances are then represented on a two-dimensional Shepard diagram scatterplot which facilitates visualization and the interpretation of patterns.

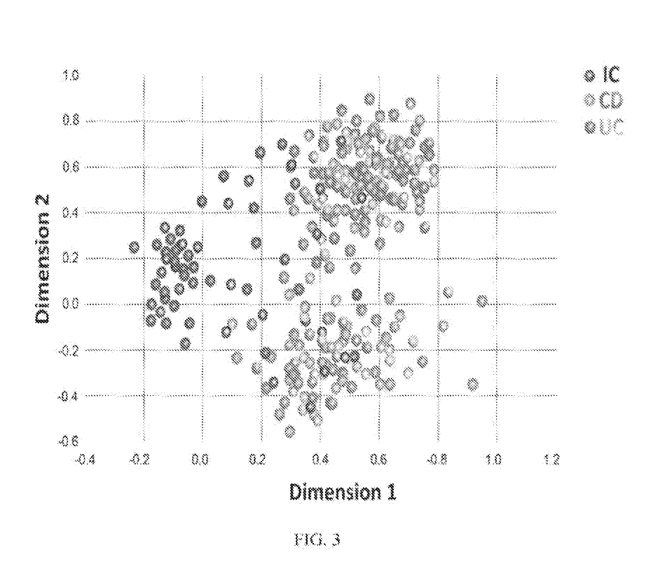

As shown in FIG. 3, MDS identified strong positive clusters of subgroups of IC, UC and CD (r=0.619 to 0.874, p<0.05). Not surprisingly, MDS also identified subgroups of IC that strongly clustered with both CD and UC respectively (r=0.596 to 0.7, p<0.05). These unique representations provide a visual inspection of similarities and differences between IC and IBD, indicating the intricate but distinct disease profiles associated with cytokine patterns.

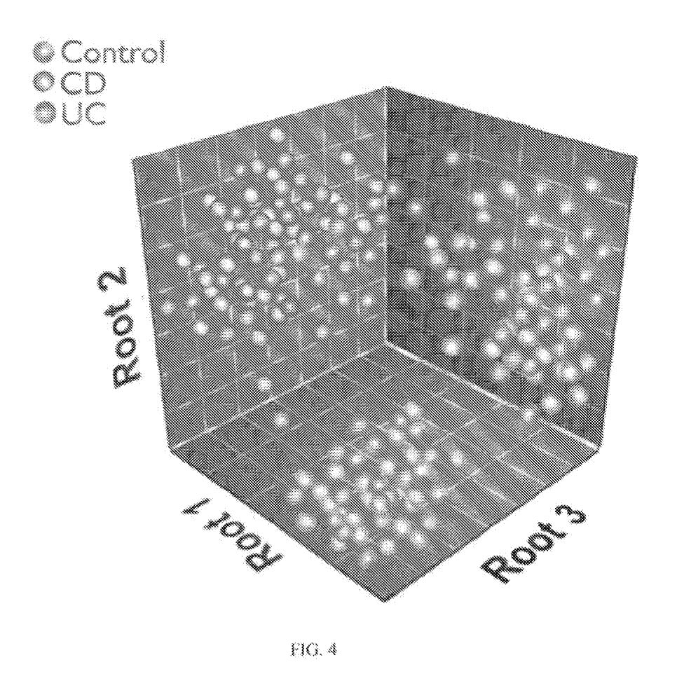

DFA was used to identify the cytokines that best discriminated between IBD and controls, and was modeled as described above. Variables were continued to be included in the model as long as they remained statistically significant. The discriminant potential of the final equation from the forward stepwise DFA could then be observed in a simple multidimensional plot of the values of the roots obtained for each group of UC, CD and controls, as represented in FIG. 4. This multivariate approach further validated the distinctive cytokine profiles, the changes of which in levels can delineate profiles and create diagnostic patterns.

Data sets were also analyzed by principal components analysis (PCA). PCA is a data reduction technique which transforms data via a linear combination to uncorrected orthogonal variables (principal components), allowing sources of variation in the data to be categorized. A PCA with Varimax rotation was used to uncorrelate the cytokines and a cold-deck imputation of the lowest quantitatable standard was used for low thresholds. If any cytokine required more than 20% imputation, the cytokine was dropped from further analysis. This analysis provided a minimum threshold allowing computation if cytokines reached the threshold. The dataset was then randomly split into 2 datasets: a test and a train dataset. The train dataset was used to construct our model, while the test dataset provides independent validation of our model. An eigenvalue greater than 1 was used to retain principal components. Once new variables were created the resulting components were placed into a logistic regression to create the predictive model of IBD. This model enabled the definition of specific parameters for diagnosis and prognosis in IBD.

### Example 3

### Development of a Cytokine Level Securing System

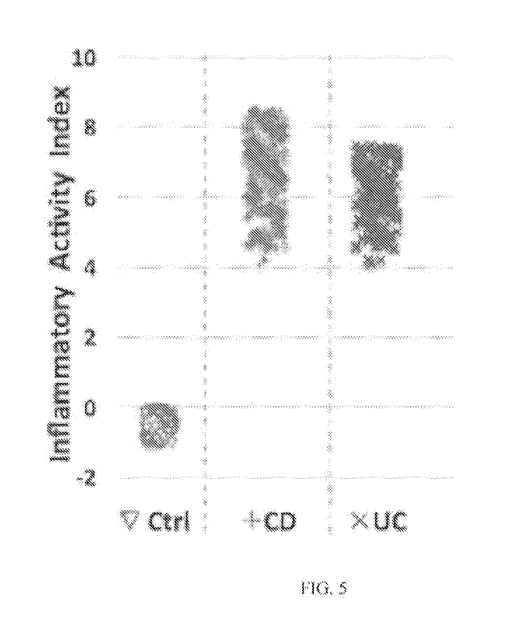

Finally, to develop an index of cytokine levels using the results of the multivariate analysis that could be readily interpreted in a clinical context for the medical community, a new scoring system was created, denoted the Inflammatory Activity Index (IAI). These values represent the levels deduced from an algorithm containing aggregate of relevant cytokine measurements where the root values from the discriminant functional analysis were normalized such that the maximal value of controls was 0. The normal IAI range was calculated in a standard manner, i.e. normal range=the 25-75% interquartile range of unaffected control values. FIG. 5 denotes a graphical representation of IAI values measured, and shows that all patient values for both UC and CD were elevated relative to unaffected controls (sensitivity>0.93).

As described herein, this is the first time that profiling, as applied particularly to cytokines, has been used to diagnose, and correlate with disease activity in IBD. The clinical assessment tools that could be derived from this approach may provide a means to (a) diagnose, (b) assess disease inflammatory activity, and (c) continually track patients such that therapeutic strategies can be better evaluated on a patient-specific basis.

### REFERENCES

1. Xavier et al., 448 NATURE 427-34 (2007).
2. Strober et al., 117 J. CLIN. INVEST. 514-21 (2007).
3. Sartor, R. B., NAT. CLIN. PRACT. GASTROENTEROL. HEPATOL. 390-407 (2006).
4. Geboes, et al., 9 INFLAMM. BOWEL. DIS. 324-31 (2003).
5. Jess et al., 12 INFLAMM. BOWEL. DIS. 669-76 (2006).
6. Sanchez-Munoz et al., 14 WORLD J. GASTROENTEROL. 4280-88 (2008).
7. Kolls et al., 8 NAT. REV. IMMUNOL. 829-35 (2008).
8. Andoh et al., 14 World J. Gastroenterol. 5154-61 (2008).
9. Fantini et al., 13 Inflamm. Bowel Dis. 1419-23 (2007).
10. Nishimura et al., 1173 Ann. K Y. Acad. Sci. 350-56 (2009).
10. O'Shea et al., 28 Immunity 477-87 (2008).
12. Furuzawa-Carballeda et al., 6 Autoimmun. Rev. 169-75 (2007).
13. Bettelli et al., 453 Nature 1051-57 (2008).
14. Charo et al., 354 N. Engl. J. Med. 610-21 (2006).
15. Zlotnik et al., 7 Genome Biol. 243 (2006).
16. Pizarro et al., 58 Ann. Rev. Med. 433-44 (2007).
17. Pizarro et al., 55 Gut 1226-27 (2006).
18. Kunz et al., 2009 Mediators Inflamm. 1-20 (2009).
19. Dryden, G. W., 9 Expert Opin. Biol. Ther. 967-74 (2009).
20. Rutgeerts et al., 136 Gastroenterology 1182-97 (2009).
21. Li et al., 14 World J. Gastroenterol. 5115-24 (2008).
22. Alex et al., 15 Inflamm. Bowel Dis. 341-52 (2009).
23. Simren et al., 97 Am. J. Gastroenterol. 389-96 (2002).
24. Quigley, E. M., 6 Chin. J. Dig. Dis. 122-32 (2005).

1-23. (canceled)

24. A method comprising the step of measuring the levels of a panel of cytokines comprising IL-1β, IL-1F3, IL-2, IL-4, IL-5, IL-6, IL-8, IL-10, IL-12, IL-17, IFN-γ, G-CSF, Eotaxin and CXCL10, in a sample collected from a patient using an immunoassay.

25. The method of claim 24, wherein the patient has Crohn's Disease (CD), Ulcerative Colitis (UC), or Indeterminate Colitis (IC).

26. The method of claim 24, wherein the patient sample comprises peripheral blood, serum plasma, cerebrospinal fluid, tissue sample, skin or other body fluid.

27. A method comprising the step of testing a patient sample from a patient that has Inflammatory Bowel Disease (IBD) to determine levels of a group of cytokines consisting of IL-1β, IL-1F3, IL-2, IL-4, IL-5, IL-6, IL-8, IL-10, IL-12, IL-17, IFN-γ, G-CSF, Eotaxin and CXCL10, wherein the patient sample is contacted with a group of antibodies which specifically bind the group of cytokines.

28. The method of claim 27, wherein the patient sample comprises peripheral blood, serum plasma, cerebrospinal fluid, tissue sample, skin or other body fluid.
